(19) United States
(12) Patent Application Publication
(10) Pub. No.: US 2024/0050525 A1
(43) Pub. Date: Feb. 15, 2024

(54) METHODS OF TREATING, DIAGNOSING AND PREDICTING PROGNOSIS OF SEPSIS

(71) Applicant: University of Rochester, Rochester, NY (US)

(72) Inventors: Anthony P. PIETROPAOLI, Pittsford, NY (US); Minsoo KIM, Pittsford, NY (US); Alissa TRZECIAK, New York, NY (US)

(21) Appl. No.: 18/259,092

(22) PCT Filed: Jan. 31, 2022
(86) PCT No.: PCT/US22/14480

§ 371 Date: Jun. 23, 2023

Related U.S. Application Data
(60) Provisional application No. 63/200,147, filed on Feb. 17, 2021.

(51) Int. Cl. A61K 38/17; G01N 33/68; A61K 9/00; A61P 37/00

(52) U.S. Cl. CPC A61K 38/1725; G01N 33/6893; A61K 9/0073; A61P 37/00; G01N 2800/26; G01N 2333/4716; G01N 2800/52

(57) ABSTRACT

A method of treating sepsis or a sepsis-related condition, comprising the step of: administering to a subject in need of such treatment an effective amount of C1q, wherein the subject is diagnosed with sepsis and wherein C1q protein expression in neutrophils of the subject is below a threshold level. A method of determining sepsis with poor prognosis in a subject, said method comprising: isolating neutrophils from the subject; determining a level of C1q protein in the isolated neutrophils, and administering C1q to the subject if the level of C1q protein is below a pre-determined threshold. Kits and compositions for carrying out methods of treatment and diagnosis are also disclosed.

This application is a national stage of International Application No. PCT/US2022/014480, filed Jan. 31, 2022, which claims priority of U.S. Provisional Application No. 63/200,147, filed Feb. 17, 2021. The entirety of the aforementioned applications is incorporated herein by reference.

### FIELD

This application relates generally to medical treatment and diagnosis and, in particular, to the prognosis and treatment of sepsis.

### BACKGROUND

Sepsis is the body's extreme response to an infection. It is a life-threatening medical emergency. Sepsis happens when an infection triggers a harmful chain reaction throughout the s body. Without timely diagnosis, triage, and treatment, sepsis can rapidly lead to tissue damage, organ failure, and death.

Almost any type of infection can lead to sepsis. Infections that lead to sepsis most often start in the lung, urinary tract, kidney, skin, or gastrointestinal tract. Most sepsis is caused by bacterial infections. It can also be a result of other infections, including viral infections such as COVID-19 or influenza, fungal infections and protozoal infections.

Previously, a sepsis diagnosis required the presence of at least two systemic inflammatory response syndrome (SIRS) criteria in the setting of presumed infection. These criteria are used to screen infected patients and identify those with sepsis. In 2016, a shortened sequential organ failure assessment score (SOFA score), known as the quick SOFA score (qSOFA), was proposed to replace the SIRS system of screening. qSOFA screening criteria for sepsis include at least two of the following three: increased breathing rate, change in the level of consciousness, and low blood pressure. Sepsis guidelines recommend obtaining blood cultures before starting antibiotics; however, the diagnosis does not require the blood to be infected. Medical imaging is helpful when looking for the possible location of the infection. Both sets of screening criteria noted above are more sensitive than specific for identifying sepsis. Some of the other potential causes of similar signs and symptoms include expected reactions to surgery, heart failure, anemia, dehydration, pulmonary embolism, anaphylaxis, and adrenal insufficiency.

Severe sepsis is defined as the systemic inflammatory response to infection that causes vital organ dysfunction. Although sepsis is one of the most common reasons for hospital death, patient response to the disease is highly heterogeneous and it is often difficult to identify the those patients who will progress to multi-system organ failure and death. However, optimal advancement of sepsis management requires precise prognostic measures in order to properly triage patients. In addition, true therapeutic breakthroughs will require accurate and relevant biomarkers identifying sub-groups of patients who will benefit from novel treatments targeted to the pathophysiologic pathway signaled by that biomarker.

### SUMMARY

One aspect of the present application relates to a method of treating sepsis or a sepsis-related condition, comprising the step of: administering to a subject in need of such treatment an effective amount of C1q protein or a variant thereof.

In some embodiments, the subject is diagnosed with sepsis and wherein C1q protein expression in neutrophils of the subject is below a threshold level.

In some embodiments, the method further comprises the step of administering to the subject an antibiotic.

In some embodiments, the C1q protein or a variant thereof is administered by inhalation, intra-tracheal delivery, nebulized and inhaled delivery, intraurethal delivery, intravenous injection or combinations thereof. In some embodiments, the C1q protein or a variant thereof is administered in an amount of 0.1-10 mg/kg body weight daily for a period of 1-14 days. In some embodiments, the C1q protein or a variant thereof is administered in an amount of 0.3-3 mg/kg body weight daily for a period of 2-7 days. In some embodiments, the C1q protein or a variant thereof is administered in an amount of 0.1-10 mg/kg body weight three time a day by local injection or intravenous injection or a combination of both for a period of 2-7 days.

In some embodiments, C1q protein or a variant thereof is administered by inhalation for treatment of sepsis caused by lung infection. In some embodiments, the C1q protein or a variant thereof is administered by retrograde infusion from bladder for treatment of sepsis caused by kidney infection. In some embodiments, the C1q protein or a variant thereof is administered by intraurethral delivery for treatment of sepsis caused by bladder infection.

In some embodiments, the sepsis-related condition is pneumonia, pneumonitis, urinary tract infection, peritonitis, infections of the biliary system including cholecystitis, cholangitis, colitis, enteritis, bowel obstruction, bowel perforation, bloodstream infection, meningitis or encephalitis, cellulitis or other skin/soft tissue infection, prostatitis, endometritis, and post-operative wound infections. Table 1 provides a list of sepsis-related conditions that may be treated with C1q administration.

TABLE 1
Sepsis-related conditions in human subjects
Organ dysfunction

| Organ | conditions |
|---|---|
| Cardiovascular | Systolic blood pressure < 90 mm Hg or a decline of >40 mm Hg, mean |
| | arterial pressure < 70 mm Hg, or requirement for intravenous |
| | vasopressor or inotropic support |
| Respiratory | Acute need for mechanical ventilation or PaO2:FiO2 ratio ≤ 250 in the |
| | absence of pneumonia (≤200 in the presence of pneumonia) |
| Hematologic | Platelet count < 100,000 or coagulopathy (international normalized |
| | ratio > 1.5) |
| Renal | Urine output < 0.5 ml/kg/hour for >2 hours despite adequate fluid |
| | resuscitation, creatinine increase of >0.5 mg/dL, or creatinine > 2.0 |
| | mg/dL |
| Neurological | Acute alteration in mental status |
| Acid/base | Lactate above upper normal laboratory limit |
| Hepatic | Bilirubin > 2 mg/dL |

Infection
Polymorphonuclear cells in a normally sterile site
Culture of pathogenic organism from normally sterile site
Chest x-ray changes consistent with pneumonia
Focus of infection identified visually
Underlying disease or condition known to be associated with infection (e.g., ascending cholangitis)

Another aspect of the present application relates to a method of determining sepsis with poor prognosis in a subject. The method comprises the steps of isolating neutrophils from the subject; determining a level of C1q protein in the isolated neutrophils, and administering C1q protein or a variant thereof to the subject, if the level of C1q protein is below a pre-determined threshold.

In some embodiments, the neutrophils are isolated from the blood of the subject. In some embodiments, the neutrophils are isolated from the blood of the subject by centrifugation. In some embodiments, the level of C1q is determined by ELISA. In some embodiments, the subject is a mammal.

Another aspect of the present application relates to a kit for providing a prognosis of a subject with sepsis. The kit comprises a test device for determining a level of C1q in a test sample; and a user guide.

In some embodiments, the test device is a testing strip that provides a visible signal when in contact with the test sample and when the level of C1q in the test sample is above a threshold level, and wherein a negative test result indicates a poor prognosis. In some embodiments, the kit further comprises a blood collection tube. In some embodiments, the kit further comprises a cell lysis reagent.

Another aspect of the present application relates to a pharmaceutical composition. The pharmaceutical composition comprises a recombinant C1q protein or a variant thereof; and a pharmaceutically acceptable carrier, wherein the pharmaceutical composition is formulated for inhalation.

### BRIEF DESCRIPTION OF THE DRAWINGS

FIG. 1 shows traditional sepsis biomarkers do not predict mortality, but CD49c+ neutrophil subpopulation can. Panel A: Study flow diagram. Neutrophils from severe sepsis patients within 48 hours of meeting diagnostic criteria (D<2), and two subsequent samples obtained at 3-5 day intervals (n=D<2: 55, D5-7: 43, D7-10: 24, age 55.5±5 years, source of infections: renal, lung, abdomen, genitourinary) were isolated. Panel B: Diagnosis of sepsis survival and death. Panels C and D: Sepsis, and serum factors (C) and neutrophil markers (D) were measured. The graph shows the absolute values or fold-increase of MFI over D<2 (Mann-Whitney U test). Panel E: Heatmap of gene expressions. Panel F: PCA of differentially expressed genes in neutrophil subsets (CD49chigh vs. CD49clow) from septic patients. Panel G: Differentially expressed genes were analyzed and pathway enrichment is expressed as the log (−P).

FIG. 2 shows C1q expression in septic neutrophils. Panel A: Comparison of differentially expressed genes in septic neutrophil subsets. Red denotes genes increased and blue denotes genes decreased in neutrophils. Panel B: PCA of differentially expressed genes in neutrophil subsets in endotoxemic mice or naïve (PBS) mice. Panels C and D: Heatmap of gene expressions (z-score), left. The top 25 genes with the lowest p value (p<10-10) were the complement pathway related genes, right. Panel E: C1q expression in human neutrophil. Panel F: Total C1q secretion in the serum and peritoneum during sepsis in mice at 6 and 12 h as measured by ELISA. Panel G: Neutrophil count in the mouse peritoneum. Panel H: Expression of C1q in mouse neutrophils after stimulation. Panel I: qPCR of C1qa, C1qb, and C1qc in healthy and sepsis patients as compared to three averaged housekeeping genes. Panel J: Deceased septic patients failed to express C1q protein in neutrophils. C1q western blot analysis on neutrophils isolated from healthy (H), deceased (D), and survived (S) sepsis patients. M; male, F; female. Panel K: Note that the serum levels of C1q did not change. Data are presented as mean+/−SEM vs. b-actin. Data were analyzed by ordinary one-way ANOVA with Tukey's multiple comparison post-test.

FIG. 3 shows blocking of C1q increases sepsis mortality. Panel A: Survival assay of LPS-induced endotoxemia mice treated with isotype IgG control antibody or anti-C1q neutralizing antibody. n=6 per group. IL-6 and IL-1b secretion in the serum (right). Panel B: Survival assay of CLP-induced septic mice treated with isotype IgG control antibody or anti-C1q neutralizing antibody. n=6 per group. IL-6 and IL-1b secretion in the serum (right). Panel C: Survival curves and sepsis severity of WT and C1q cKO mice treated with LPS, n=6 per group. Data are presented as mean+/−SEM. Data were analyzed by two-way ANOVA. (*P<0.05; **P<0.01). Panel D: IL-6, IL-1b, and IL-10 secretion in the serum. Panel E: Flow cytometric analysis of apoptotic neutrophils in the peripheral organs (lung+kidney+peritoneum) after LPS treatment (mean±s.e.m., n=4 mice per group). Panel F: Tissue injury was severe in C1q cKO mice. The representative H&E staining lung sections are shown.

FIG. 4 shows: Panel A: Neutrophil-specific C1q expression is essential for proper efferocytosis of apoptotic neutrophils. Apoptotic neutrophils secrete C1q and “self-decorate” in an autocrine and/or paracrine fashion, marking them for efficient efferocytosis by local resident macrophage, leading to resolution of infection and survival. Without C1q, apoptotic neutrophils build up causing prolonged inflammation and therefore poorer prognosis and eventually, death. Panel B: Apoptotic neutrophils secret C1q. C1q secretion by neutrophils in response to TNF (2-200 ng ml-1), fMLP (0.1-10 μM), or FasL (1-100 ng ml-1) stimulation was determined by Western blot analysis of neutrophil supernatants. Each panel shows one representative image of three replicated experiments. Data were analyzed by two-way ANOVA. (*P<0.05). Panel C: C1q binds to apoptotic neutrophils and promote phagocytosis. Live or apoptotic human neutrophils were incubated with C1q. C1q binding was measured by flow cytometry using anti-human C1q Ab. Data obtained in three experiments using different donors. (*P<0.05) Student's t test. Panel D: Phagocytosis of apoptotic human neutrophils by U937 cells was measured with CypHer 5 in the presence of C1q. Panel E: Entrapment of neutrophil inside the pulmonary capillary of endotoxic mice. Real-time imaging of Ly6G+ (red) neutrophil entrapment in the pulmonary capillary (dextran, blue) after LPS treatment. Panel F. Injection of purified C1q ameliorates the neutrophil accumulation in the lung of LPS-treated mice. Right: Comparison of number of Ly6G+ cells in pulmonary microcirculation between the PBS, LPS, LPS+C1q groups (10 fields of view per mouse, three mice per group, two-tailed t-test, *p<0.01). Data are presented as mean±SEM. Panels G and H: Survival curves and sepsis severity (right) of mice treated with LPS (G) or CLP (H)+/−serum purified C1q, n=6 per group. (right) Data are presented as mean+/−SEM. Data were analyzed by Mantel-Cox for A and two-way ANOVA. Panel I: mRNA expression in the mouse septic macrophage for potential C1q receptors.

While the present disclosure will now be described in detail, and it is done so in connection with the illustrative embodiments, it is not limited by the particular embodiments illustrated in the figures and the appended claims.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### Definitions and Terminology

As used herein, the following terms shall have the following meanings:

As used in this specification and the appended claims, the singular forms “a,” “an” and “the” include plural referents unless the content clearly dictates otherwise.

The term “C1q protein,” as used herein, refers to the complement component 1q. C1q together with C1r and C1s form the C1 complex, which initiates the conventional pathway of activation of the complement system. The binding of C1q to “danger signals”, namely antigen-antibody complexes and factors present at the surface of pathogenic agents, of infected cells or of apoptotic cells, results in the autoactivation of C1r, which activates C1s. Activated C1s then initiates the activation in cascade of the other complement components. References to “C1q” herein incorporate by reference polypeptide variants, homologous sequences, fragments and other amino acid sequences having sequence identity to C1q protein.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “variant” refers to protein or polypeptide that is different from the reference protein or polypeptide by one or more amino acids, e.g., one or more amino acid substitutions, but substantially maintains the biological function of the reference protein or polypeptide. The term “variant” further includes conservatively substituted variants. The term “conservatively substituted variant” refers to a peptide comprising an amino acid residue sequence that differs from a reference peptide by one or more conservative amino acid substitution, and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another, such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. The phrase “conservatively substituted variant” also includes peptides wherein a residue is replaced with a chemically derivatized residue, provided that the resulting peptide maintains some or all of the activity of the reference peptide. In some embodiments, the functional variant of a peptide shares a sequence identity of 70%, 75%, 80%, 85%, 90%, 91%, 92%, 93%, 94%, 95%, 96%, 97%, 98% or 99% with the reference peptide. For example, a functional variant of a protein may share a sequence identity of 70%, 75%, 80%, 85%, 90%, 91%, 92%, 93%, 94%, 95%, 96%, 97%, 98% and 99% with the reference version of the protein; and a functional variant of a fusion protein may shares a sequence identity of 70%, 75%, 80%, 85%, 90%, 91%, 92%, 93%, 94%, 95%, 96%, 97%, 98% and 99% with the reference fusion protein.

A variant of a polypeptide may be a fragment of the original polypeptide. The term “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 3, 5, 6, 8 or 10 amino acids long, at least 14 amino acids long, at least 20, 30, 40 or 50 amino acids long, at least 75 amino acids long, or at least 100, 150, 200, or more amino acids long.

The term “homologous amino acid sequence” used in this specification, unless otherwise stated herein, refers to an amino acid sequence derived from the substitution of one or more amino acids in the amino acid sequence of a polypeptide. Furthermore, the term “homologous polypeptide” used in this specification, unless otherwise stated herein, refers to a polypeptide homologue derived from the substitution of one or more amino acids in the amino acid sequence of a polypeptide.

The term “sequence identity,” as used herein, means that two peptide sequences are identical (i.e., on an amino acid-by-amino acid basis) over the window of comparison. The term “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. The reference sequence may be a subset of a larger sequence, for example, as a segment of the full-length sequences of the compositions claimed in the present invention.

The term “sepsis” refers to a bloodstream infection with highly heterogeneous presentation, progression, and high mortality. Sepsis occurs when chemicals released in the bloodstream to fight an infection trigger a maladaptive inflammatory response in the body. This can cause a cascade of changes that damage multiple organ systems, leading them to fail, sometimes even resulting in death. Symptoms include fever, difficulty breathing, low blood pressure, fast heart rate, and mental confusion. Treatment includes antibiotics and intravenous fluids.

A patient presenting with sepsis is a patient who manifests the clinical criteria used to identify a patient with sepsis, symptoms may include fever, difficulty breathing, low blood pressure, fast heart rate, and mental confusion. For example, a sepsis diagnosis requires the presence of infection, which can be proven or suspected, and 2 or more of the following criteria: hypotension (systolic blood pressure<90 mm Hg or fallen by >40 from baseline, mean arterial pressure<70 mm Hg); Lactate>1 mmol/L; mottled skin; Decreased capillary refill of nail beds or skin; Fever>38.0° C., or 101° F.; Hypothermia<36° C. core temperature (<96.8° F.); Heart rate>90; Tachypnea; Change in mental status; Significant edema or positive fluid balance (>20 mL/kg over 24 hours); Hyperglycemia (>140 mg/dL) in someone without diabetes; White blood cell count>12,000 or less than 4,000, or with >10% “bands” (immature forms); elevated C-reactive protein in serum (>10 mg/L); elevated procalcitonin in serum (>2 ng/mL); arterial hypoxemia (paO2/FiO2<300); acute drop in urine output (<0.5 ml/kg/hr for at least 2 hours despite fluid resuscitation, or about 35 ml/hour for a 70 kg person); creatinine increase>0.5 mg/dL; international normalized ratio (INR)>1.5 or activated partial thromboplastin time (aPTT)>60 seconds; absent bowel sounds (ileus); platelet count<100,000; high bilirubin (total bilirubin>4 mg/dL).

The term “severe sepsis” refers to sepsis with impaired blood flow to body tissues (hypoperfusion) or detectable organ dysfunction. Severe sepsis may occur with or without sepsis-induced hypotension (e.g., with fever, encephalopathy and renal failure but a normal blood pressure).

The term “septic shock” refers to severe sepsis with sepsis-induced hypotension (systolic blood pressure<90 mm Hg (or a drop of >40 mm Hg from baseline) or mean arterial pressure<70 mm Hg) that persists after adequate fluid resuscitation. “Adequate” is determined by the estimation of the patient's intravascular volume status.

The term “systemic inflammatory response syndrome,” or “SIRS,” refers to a clinical response to a variety of severe clinical insults, as manifested by two or more of the following conditions within a 24-hour period: body temperature greater than 38° C. (100.4° F.) or less than 36° C. (96.8° F.); heart rate (HR) greater than 90 beats/minute; respiratory rate (RR) greater than 20 breaths/minute, or PCO2 less than 32 mmHg, or requiring mechanical ventilation; and white blood cell count (WBC) either greater than 12.0×109/L or less than 4.0×109/L or having greater than 10% immature band forms.

These symptoms of SIRS represent a consensus definition of SIRS that can be modified or supplanted by other definitions in the future. The present definition is used to clarify current clinical practice and does not represent a critical aspect of the application (see, e.g., American College of Chest Physicians/Society of Critical Care Medicine Consensus Conference: Definitions for Sepsis and Organ Failure and Guidelines for the Use of Innovative Therapies in Sepsis, 1992, Crit. Care. Med. 20, 864-874, the entire contents of which are herein incorporated by reference).

A subject with SIRS has a clinical presentation that is classified as SIRS, as defined above, but is not clinically deemed to be septic. Methods for determining which subjects are at risk of developing sepsis are well known to those in the art. Such subjects include, for example, those in an intensive care unit (ICU) and those who have otherwise suffered from a physiological trauma, such as a burn, surgery or other insult. A hallmark of SIRS is the creation of a proinflammatory state that can be marked by tachycardia, tachypnea or hyperpnea, hypotension, hypoperfusion, oliguria, leukocytosis or leukopenia, pyrexia or hypothermia and the need for volume infusion. The “onset of sepsis” refers to an early stage of sepsis, e.g., prior to a stage when conventional clinical manifestations are sufficient to support a clinical suspicion of sepsis. Because the methods of the present application can be used to detect sepsis prior to a time that sepsis would be suspected using conventional techniques, in certain embodiments, the subject's disease status at early sepsis is confirmed retrospectively, when the manifestation of sepsis is more clinically obvious. The exact mechanism by which a subject becomes septic is not a critical aspect of the application. The methods of the present application can detect the onset of sepsis independent of the origin of the infectious process.

The term “diagnosis,” as used herein, generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

The term “prognosis,” as used herein, generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

A “clinical marker” refers to a physiological parameter that can be measured in the subject, such as a clinical vital sign. Examples include, but are not limited to respiratory rate, temperature, heart rate, systolic blood pressure, diastolic blood pressure mean artery pressure, white blood cell count, monocyte count, lymphocyte count, granulocyte count, neutrophil count, immature neutrophil to total neutrophil ratio, platelet count, serum creatinine concentration, urea concentration, lactate concentration, glucose concentration, base excess, pO2 and HCO3-concentration.

A “biomarker” is a compound that is present in or derived from a biological sample. The phrase “derived from,” as used in this context, refers to a compound that, when detected, is indicative of a particular molecule being present in the biological sample. For example, detection of a particular fragment of a compound can be indicative of the presence of the compound itself in the biological sample. A biomarker can, for example, be isolated from the biological sample, directly measured in the biological sample, or detected in or determined to be in the biological sample. A biomarker can, for example, be functional, partially functional, or non-functional.

As used herein, the term “subject” includes both human and animal subjects. Thus, veterinary therapeutic uses for pets, zoo animals and livestock are provided in accordance with the presently disclosed subject matter.

The term “mammal” refers to any animal classified as a mammal, including humans, non-human primates, domestic and farm animals, and zoo, sports, or pet animals, such as dogs, horses, cats, cows, etc. Preferably, the mammal is human.

A “therapeutically effective amount,” as used herein, refers to an amount effective, at dosages and for periods of time necessary, to achieve the desired therapeutic result. A “prophylactically effective amount” refers to an amount effective, at dosages and for periods of time necessary, to achieve the desired prophylactic result.

As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

### Method of Treating Sepsis or a Sepsis-related Condition

The authors of this application have unexpectedly discovered that low C1q protein levels in neutrophils of sepsis patient are associated with low survival rate, and that the rate of survival increases if septic animals are given the exogenously expressed C1q protein. Accordingly, one aspect of the present application relates to a method of treating sepsis or a sepsis-related condition, comprising the step of: administering to a subject in need of such treatment a therapeutically effective amount of C1q protein or a variant thereof. Examples of sepsis-related conditions include, but are not limited to pneumonia, pneumonitis, urinary tract infection, peritonitis, infections of the biliary system including cholecystitis, cholangitis, colitis, enteritis, bowel obstruction, bowel perforation, bloodstream infection, meningitis or encephalitis, cellulitis or other skin/soft tissue infection, prostatitis, endometritis, and post-operative wound infections.

### C1q Protein

Circulating C1q protein is a 400 kDa protein complex composed of 18 polypeptide chains: six A-chains, six B-chains, and six C-chains. The complete amino acid sequences of the A, B and C chain of the human C1q protein are listed in SEQ ID NOS:1-3. The assembled C1q hexamer contains a central core or stalk, 6 collagen-like domains and 6 globular protein heads. These globular or terminal regions are responsible for the binding of immunoglobulins (IgM, and IgG). C1q is a subunit of the C1 enzyme complex that activates the serum complement system.

In some embodiments, the C1q protein is a C1q protein from a mammalian species. In some embodiments, the C1q protein is a wild type human C1q protein, or a variant of a human C1q protein. In some embodiments, the C1q protein is a purified human C1q protein. In some embodiments, the human C1q is produced in human plasma.

In some embodiments, the C1q protein is a recombinant protein. A recombinant C1q protein may be prepared in accordance with U.S. Pat. No. 10,294,284, which is incorporated herein by reference.

In some embodiments, the C1q protein is a chemically modified C1q protein with improved pharmacokinetics, stability, and/or therapeutic activity. Examples of the chemically modified C1q protein include, but are not limited to, C1q proteins modified by PEGylation, glycosylation and/or mannosylation. PEGylation of the C1q protein alters the solubility, size, molecular weight, and steric hindrance of the C1q protein. In some embodiments, the C1q protein is modified by a non-degradable PEG alternative. Examples of non-degradable PEG alternatives include, but are not limited to, Poly(vinyl pyrrolidone) (PVP) and Poly(N-(2-hydroxypropyl)methacrylamide) (PHPMA), Polyglycerol (PG), Polyoxazolines (POZs), and Poly(N-acryloylmorpholine) (PNAM). Degradable PEG alternatives include Polysialic acid (PSA), Trehalose glycopolymers, Hydroxyethyl starch (HES), poly(ethyl ethylene phosphate) (PEEP) and Recombinant synthetic polypeptides. Glycosylated and mannosylated C1q proteins may demonstrate different pharmacological properties than PEGylated C1q protein.

In some embodiments, the C1q protein is conjugated to a conjugation partner. Examples of the conjugation partner include, but are not limited to, bile acid transporters, amino acid and oligopeptide transporters, water-soluble vitamin transporters, phosphate transporters, monocarboxylic acid transporters and carbohydrate transporters. In some embodiments, the C1q protein is covalently conjugated to the conjugation partner, such as a FLAG-tag or Fc-tag.

### Dosage and Treatment Regimen

The amount of C1q protein to be administered may be determined based on the need of the subject. In some embodiments, the C1q protein is administered in a sufficient amount to facilitate clearance of infiltrated neutrophils in a target tissue or organ.

In some embodiments, the C1q protein is administered, individually or in combination with other agents, in one or more doses in the dose range of 0.01-100 mg/kg body weight, 0.01-30 mg/kg body weight, 0.01-10 mg/kg body weight, 0.01-3 mg/kg body weight, 0.01-1 mg/kg body weight, 0.01-0.3 mg/kg body weight, 0.01-0.1 mg/kg body weight, 0.01-0.03 mg/kg body weight, 0.03-100 mg/kg body weight, 0.03-30 mg/kg body weight, 0.03-10 mg/kg body weight, 0.03-3 mg/kg body weight, 0.03-1 mg/kg body weight, 0.03-0.3 mg/kg body weight, 0.03-0.1 mg/kg body weight, 0.1-100 mg/kg body weight, 0.1-30 mg/kg body weight, 0.1-10 mg/kg body weight, 0.1-3 mg/kg body weight, 0.1-1 mg/kg body weight, 0.1-0.3 mg/kg body weight, 0.3-100 mg/kg body weight, 0.3-30 mg/kg body weight, 0.3-10 mg/kg body weight, 0.3-3 mg/kg body weight, 0.3-1 mg/kg body weight, 1-100 mg/kg body weight, 1-30 mg/kg body weight, 1-10 mg/kg body weight, 1-3 mg/kg body weight, 3-100 mg/kg body weight, 3-30 mg/kg body weight, 3-10 mg/kg body weight, 10-100 mg/kg body weight, 10-30 mg/kg body weight or 30-100 mg/kg body weight.

In some embodiments, the C1q protein is administered, individually or in combination with other agents, in one or more doses in the range of 0.6-6000 mg/dose, 0.6-2000 mg/dose, 0.6-1000 mg/dose, 0.6-300 mg/dose, 0.6-100 mg/dose, 0.6-30 mg/dose, 0.6-10 mg/dose, 0.6-3 mg/dose, 2-6000 mg/dose, 2-2000 mg/dose, 2-1000 mg/dose, 2-300 mg/dose, 2-100 mg/dose, 2-30 mg/dose, 2-10 mg/dose, 6-6000 mg/dose, 6-2000 mg/dose, 6-1000 mg/dose, 6-300 mg/dose, 6-100 mg/dose, 6-30 mg/dose, 20-6000 mg/dose, 20-2000 mg/dose, 20-1000 mg/dose, 20-300 mg/dose, 20-100 mg/dose, 60-6000 mg/dose, 60-2000 mg/dose, 60-1000 mg/dose, 60-300 mg/dose, 200-6000 mg/dose, 200-2000 mg/dose, 200-1000 mg/dose, 600-6000 mg/dose or 600-2000 mg/dose.

Dosage-unit form, as used herein, refers to physically discrete units suited as unitary dosages for the subjects to be treated, each unit containing a predetermined quantity of active material calculated to produce the desired therapeutic effect in association with the required pharmaceutical carrier. The specification for the dosage-unit forms of the present application can be chosen based upon: (a) the unique characteristics of the active material and the particular therapeutic effect to be achieved, and (b) the limitations inherent in the art of compounding such an active material for the treatment of conditions in living subjects having a condition in which bodily health is impaired as described herein.

Dosage regimens may vary according to the needs of the subject according to the methods known to one of ordinary skill in the art. Dosage regimens may include once per day, twice per day, three times per day, four times per day, once per week, twice per week, three times per week, four times per week. Dosage regimens may include taking doses a week before onset of sepsis, six days before onset of sepsis, five days before onset of sepsis, three days before onset of sepsis, two days before onset of sepsis, one day before onset of sepsis, at onset of sepsis, one day after onset of sepsis, two days after onset of sepsis, three days after onset of sepsis, four days after onset of sepsis, five days after onset of sepsis, six days after onset of sepsis, a week after onset of sepsis, or until sepsis has abated or is in remission. Dosage regimens may cover a range of days, such as 1-2 days, 1-3 days, 1-4 days, 1-5 days, 1-6 days, 1-7 days, 1-8 days, 1-9 days, 1-10 days, 1-11 days, 1-12 days, 1-13 days, or 1-14 days. Dosage regimens may also include other agents for treatment of sepsis in conjunction with C1q protein treatment.

### Route of Administration

The therapeutically effective amount of C1q may be administered by a route suitable for the subject. Exemplary routes of administration include parenteral (e.g., intravenous, intraarterial, intramuscular, subcutaneous, intratumoral), inhalation, mucosal (e.g., nasal, sublingual, buccal, rectal, vaginal), topical (nasal, transdermal, intradermal or intraocular), intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent. Parenteral compositions may be formulated in dosage-unit form for ease of administration and uniformity of dosage as discussed herein.

In some embodiments, the C1q protein is administered by intravenous administration to a subject.

In some embodiments, the C1q protein is administered by oral inhalation or nasal spray.

In some embodiments, the C1q protein is administered by retrograde infusion from bladder.

### Subject

The subject of the treatment can be any mammal that has an immune system with complements. In some embodiments, the subject is a human. In other embodiments, the subject is a non-human primate, a zoo animal or a pet.

In some embodiments, the subject has sepsis. In some embodiments, the subject has severe sepsis. In some embodiments, the subject is in sepsis shock.

In certain embodiments, the subject is sepsis-negative. In the context of this application, sepsis-negative subjects include subjects that, for any reason according to the judgment of a practitioner of the art, are in need of the treatment of the present application. Such subjects include, but are not limited to, sepsis-negative subjects in hospital intensive care units and similarly situated subjects. In particular embodiments, the subject is a subject that is in intensive care unit and might be at risk for a systemic inflammatory condition.

In some embodiments, the subject is at a risk of developing sepsis. Further methods of the application can be used to monitor a treatment or prevention for increased or decreased likelihood of onset of severe sepsis, septic shock, multiple organ dysfunction or mortality, or to monitor for possible conversion to sepsis-positive or sepsis-negative.

### Combination Therapy

In some embodiments, the method further comprises the step of administering to the subject another treatment agent. In some embodiments, the treatment agent is an antibiotic. In some embodiments, the treatment agent is an intravenous fluid. In some embodiments, the treatment agent is a vasopressor. In some embodiments, the treatment agent is a corticosteroid. In some embodiments, the treatment agent is insulin. In some embodiments, the treatment agent is a painkiller or sedative. In some embodiment, treatment agent is an anti-viral agent.

### Method of Prognosis or Diagnosis of Sepsis

Another aspect of the present application relates to methods for providing a prognosis for a subject with a systemic inflammatory condition. Examples of systemic inflammatory conditions include, but are not limited to, systemic inflammatory response syndrome (SIRS), sepsis, severe sepsis, septic shock and multiple organ dysfunction or mortality.

In some embodiments, the present application relates to a method of determining sepsis with poor prognosis in a subject, said method comprising: isolating neutrophils from the subject; determining a level of C1q protein in the isolated neutrophils, and administering C1q to the subject if the level of C1q protein is below a pre-determined threshold.

### Isolation of Neutrophils

The neutrophils may be isolated using conventional methods known to one skilled in the art. The neutrophils may be isolated from body fluids or tissues of the subject in any manner known to one of ordinary skill in the art. In certain embodiments, the fluid or tissue of the subject is blood, plasma, saliva, serum, sputum, urine, cells, cellular extract or tissue biopsy. In some embodiments, the neutrophils are isolated from the blood of the subject. In some embodiments, the neutrophils are isolated from the blood of the subject by centrifugation.

In some embodiments, the neutrophils are isolated from the blood by the density gradient separation method. Specifically, whole blood sample is collected from the subject, mixed with an anticoagulation agent such as EDTA, citrate, and heparin, layered over a density gradient medium, and subjected to centrifugation. The neutrophil layer is collected after centrifugation. The residual erythrocytes are lysed. The neutrophils are then washed, counted, and resuspended to desired concentration. In some embodiments, isolated neutrophils are washed, counted, and resuspended to desired concentration for determination of C1q protein levels.

In some embodiments, neutrophils may be isolated by other methods known to one of ordinary skill in the art, such as, for example, flow cytometry. Commercial kits for isolation of human neutrophils are also available and may be used.

### Detection of C1q Protein Levels

Detection of the C1q protein level in isolated neutrophils can be performed with conventional methods known to a person skill in the art. In some embodiments, the neutrophils are lysed and the amount of C1q protein in the lysate is determined by ELISA, Western blot, mass spectrometry or affinity chromatograph. In some embodiments, the amount of C1q protein in the neutrophil lysate is determined on a test strip using an ELISA based system. In a particular embodiment, the detection system is an ELISA based detection system that will show a positive signal if the amount of C1q in the test sample is above a pre-determined threshold. In some embodiment, the neutrophil C1q level is determined by flow cytometry analysis.

A low amount of C1q protein in the isolated neutrophils indicates a poor prognosis for the subject with sepsis. In some embodiments, the threshold level is the pre-sepsis neutrophil C1q level in the subject. In other embodiments, the threshold level is the average neutrophil C1q level in the health individuals. The threshold level (or reference level) can be calculated according to any suitable statistical method known to those of skill in the art. It should be noted that the threshold level of neutrophil C1q may vary at different stages of sepsis development. For example, neutrophil C1q protein level generally peaks at 3-7 days after sepsis diagnosis. Accordingly, in some embodiments, the threshold level of neutrophil C1q at 3-7 days after sepsis is higher than the threshold level of neutrophil C1q before and after this period of sepsis development.

In some embodiments, a threshold level (or reference level) of neutrophil C1q expression is identified at different stages of sepsis development by consulting data available to those of skill in the art. Such data can be obtained from any source available to those of skill in the art. In some embodiments, sources can be developed with reference amounts of neutrophil C1q protein expression collected by those of skill in the art according to methods described herein.

In some embodiments, the neutrophils are obtained from the subject during day 1-10 after diagnosis of sepsis. In some embodiments, the neutrophils are obtained from the subject during day 3-7 after diagnosis of sepsis.

In some embodiments, the neutrophils are obtained from the subject immediately prior to the onset of sepsis. In some embodiments, the neutrophils are obtained from the subject 12, 24, 36 or 48 hours prior to the onset of sepsis.

In some embodiments, the method further comprises the step of determining the level of a second prognosis marker in the subject. Examples of such prognosis marker include, but are not limited to, Lactate, procalcitonin, WBC count, ANC, CD64, CD49c, inflammatory cytokines, such as IL6, TNF, IL8, and severity of illness scoring systems, such as APACHE, SOFA, Murray Lung Injury Score, and SAPS.

In specific embodiments of the application, a neutrophil C1q protein expression profile may be determined, for example, by detecting the amount of one of the subunits of the C1q complex (e.g., the A chain, B chain or C chain of the C1q complex).

In certain embodiments, the steps of isolating neutrophils from the subject; and determining a level of C1q protein in the isolated neutrophils, are repeated one or more times at different time points; and a prognosis is made based on neutrophil C1q protein levels at the different time points.

In some embodiments, the neutrophil C1q level is monitored in a subject with sepsis using the method described above and C1q treatment is initiated when the neutrophil C1q level in the subject falls below the threshold level. In some embodiment, a subject at risk of sepsis is monitored soon after the subject arrives in an intensive care unit. In some embodiments, the subject is monitored daily after arriving in an intensive care unit. In some embodiments, the subject is monitored every 1 to 3 hours, 3 to 8 hours, 8 to 12 hours, 12 to 16 hours, or 16 to 24 hours after arriving in an intensive care unit.

### Other Indicators of Prognosis

In some embodiments of the application, the method for providing a prognosis to a subject with sepsis further comprises the step of monitoring one or more other indicators of sepsis prognosis. Examples of such indicators include, but are not limited to, levels of endotoxin, bacterial DNA, protein C, protein S, procalcitonin (PCT), C-reactive protein (CRP), LBP LPS-binding protein, fibrin degrading products, HLA-DR, cell surface proteins CD-14 and CD-64, E-selectin, cortisol, ACTH, surface-bound tumor necrosis factor receptor I (sTNFRI), surface-bound tumor necrosis factor receptor II (sTNF-RII), TNF-α, interleukins IL-6, IL-8 and IL-10, D-dimer, prothrombin, antithrombin III, activated partial thromboplastin, plasminogen activator inhibitor-1, soluble thrombomodulin, thrombin activatable fibrinolysis inhibitor, copeptin, high mobility group box 1 (HMGB1), triggering receptor expressed on myeloid cells 1 (TREM1) and albumin in the subject with sepsis.

In some embodiments, the indicators of prognosis further comprise one or more clinical indicators selected from the group consisting of respiratory rate, body temperature, heart rate, systolic blood pressure, diastolic blood pressure mean artery pressure, white blood cell count, monocyte count, lymphocyte count, granulocyte count, neutrophil count, immature neutrophil to total neutrophil ratio, platelet count, serum creatinine concentration, urea concentration, lactate concentration, glucose concentration, base excess, pO2, HCO3-concentration, and severity of illness scoring systems (such as APACHE, SOFA, Murray Lung Injury Score, and SAPS).

### Administration of Treatment

In some embodiments, if the neutrophil C1q level in the subject is below a threshold level (or reference level), the subject is treated with exogenous C1q protein or a variant of C1q protein in the manner discussed in the present application.

### Pharmaceutical Composition

Another aspect of the present application is a pharmaceutical composition, comprising: a recombinant C1q protein; and a pharmaceutically acceptable carrier, wherein the pharmaceutical composition is formulated for inhalation. The C1q may be administered in any suitable form and in any suitable composition to subjects. The composition(s) may be formulated to include, for example, a fluid carrier/solvent (a vehicle), a preservative, one or more excipients, a coloring agent, a flavoring agent, a salt(s), an anti-foaming agent, and/or the like. The C1q may be present at a concentration in the vehicle that provides a prophylactically or therapeutically effective amount of the C1q for prevention or treatment of sepsis when administered to a subject at risk for developing sepsis.

A pharmaceutical composition comprising C1q in accordance with the present disclosure may be formulated in any pharmaceutically acceptable carrier(s) or excipient(s). In some embodiments, C1q can be incorporated into a pharmaceutical composition suitable for parenteral administration. In some embodiments, the pharmaceutical composition comprises a buffer. Suitable buffers include but are not limited to, sodium succinate, sodium citrate, sodium phosphate or potassium phosphate. In some embodiments, the pharmaceutical composition comprises sodium chloride at a concentration of 0-300 mM (optimally 150 mM for a liquid dosage form).

In some embodiments, polymeric micelles may be used. In further embodiments, the therapeutic C1q proteins may be delivered by polymersomes, which are composed of block or graft amphiphilic copolymers have properties similar to those of liposomes, with the advantage of a higher membrane stability. The hydrophobic domain of the polymeric membrane can incorporate hydrophobic proteins/drugs, whereas the aqueous core can encapsulate hydrophilic proteins. By varying block-copolymer composition, molecular weight and architecture, it is possible to tune the size, shape, membrane thickness, mechanical strength, permeability and surface chemistry for optimizing drug loading and delivery.

In certain embodiments, polymer networks may be used to encapsulate hydrophilic proteins within their matrix. Hydrogel nanoparticles are three-dimensional polymer networks containing a large amount of water; swelling and degradability of the hydrogel can be tuned through the choice of the type of polymer and the crosslinking density, in order to achieve an efficient protein loading and release. The polymer composition can be selected to provide stealth character, to guarantee extended plasma half-life, and to enhance targeting.

In certain embodiments, enzyme inhibitors may be added with the therapeutic C1q protein, such as sodium glycocholate, camostat mesilate, bacitaracin, soybean trypsin inhibitor and aprotinin. In certain embodiments, absorption enhancers may be used with the therapeutic C1q protein, such as chitosans, fatty acids, lectins, or zonula occludens toxin (ZOT). Other absorption enhancers, may be cell-penetrating peptides, polyamines or biliposomes.

In certain embodiments, nanoparticles may be used to deliver therapeutic C1q protein. In further embodiments, lipid-based micro- and nanocarriers such as emulsions, exosomes, non-ionic surfactant vesicles, solid lipid particles and micelles and can be used for nanoencapsulation and transport of therapeutic C1q proteins.

In particular embodiments, the therapeutic C1q proteins may be delivered by emulsions, which are colloidal dispersions composed of oil, water and surfactants. Depending on the formulation and manufacturing conditions, the oil-in-water or water-in-oil droplets can be small in size (microemulsion and nanoemulsions) and employed for the delivery of C1q proteins by non-parenteral routes, such as oral and transdermal delivery.

In further embodiments, the therapeutic C1q proteins may be delivered by exosomes, which are neutral extracellular vesicles (cell-derived vesicles) with a native membrane composition. These natural vesicles are involved in cell-to-cell communication and play an important role in the biomolecule transfer pathways. The similarities between exosomes and liposomes include the presence of the lipid bilayer (rich in cholesterol and diacylglycerol), the minimal toxicity, biocompatibility, the nanometric size and the internal volume where several biomolecules can be entrapped. The principal advantages of these nanoparticles are the high and specific organotropism and the immunocompatibility.

In certain embodiments, the therapeutic C1q proteins may be delivered by niosomes, which are non-ionic surfactant vesicles principally composed of non-ionic surfactants and cholesterol. The particle size (from 10 nm to 20 μm) depends on the preparation method and the composition. Niosomes present similar advantages of liposomes in terms of ease preparation, biocompatibility, low toxicity. In certain embodiments, the therapeutic C1q proteins may be delivered by solid lipid nanoparticles, which are composed of a solid lipid nucleus stabilized with a monolayer of phospholipids or surfactants. They are prepared using various lipids such as mono-, di- and triglycerides, phospholipids, fatty acids, waxes and steroids, and amphiphiles such as poloxamers and polysorbates.

In particular embodiments for delivery of therapeutic C1q proteins, protein lipidization is used. Conjugation of desired therapeutic substance with fatty acid increases the stability and improves the transportation of conjugated proteins across the biological membranes of gut wall. For example, caprates and triglycerides are most frequently used to increase the paracellular diffusion of proteins and prevent hepatic-metabolism respectively.

In some embodiments for delivery of therapeutic C1q proteins, colloidal carrier systems protect the C1q proteins from degradation, prolong the release rate, control the steady-state release, reduce administration frequency, maintain the plasma half-life of therapeutic C1q proteins and improve the patient compliance. Colloidal carrier systems involve several techniques including microparticles, nanoparticles, liposomes and thermosensitive gels that are known to one of ordinary skill in the art.

Therapeutic preparations can be lyophilized and stored as sterile powders, preferably under vacuum, and then reconstituted in bacteriostatic water (containing, for example, benzyl alcohol preservative) or in sterile water prior to injection. Pharmaceutical composition may be formulated for parenteral administration by injection e.g., by bolus injection or continuous infusion.

The pharmaceutical forms suitable for injectable use include sterile aqueous solutions or dispersions and sterile powders for the extemporaneous preparation of sterile injectable solutions or dispersions. The form should be sterile and fluid to the extent that easy syringability exists. It should be stable under the conditions of manufacture and storage and should be preserved against the contaminating action of microorganisms, such as bacteria and fungi. The pharmaceutical carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in the appropriate solvent with various of the other ingredients enumerated above, followed by filtered sterilization. Generally, dispersions can be prepared by incorporating the various sterilized active ingredient into a sterile vehicle containing the basic dispersion medium and the required other ingredients from those enumerated above. In the case of sterile powders for the preparation of sterile injectable solutions, methods of preparation include vacuum drying and freeze drying techniques, which yield a powder of the active ingredient plus any additional desired ingredient from previously sterile filtered solution thereof.

An effective amount of a composition disclosed herein is a nontoxic, but sufficient amount of the composition, such that the desired prophylactic or therapeutic effect is produced. The exact amount of the composition that is required will vary from subject to subject, depending on the species, age, condition of the animal, severity of the inflammation or tumor-related disorder in the animal, the particular carrier or adjuvant being used, its mode of administration, and the like. Accordingly, the effective amount of any particular therapeutic composition disclosed herein will vary based on the particular circumstances, and an appropriate effective amount can be determined in each case of application by one of ordinary skill in the art using only routine experimentation.

In some embodiments, the pharmaceutical composition is in a lyophilized dosage form and comprise a cryoprotectant. Examples of cryoprotectants include, but are not limited to, sucrose (optimally 0.5-1.0%), trehalose and lactose. In some embodiments, the pharmaceutical composition further comprises a bulking agent. Examples of bulking agents include, but are not limited to, mannitol, glycine and arginine.

Pharmaceutical compositions for treating a patient with sepsis also contain pharmaceutically or physiologically acceptable carriers, excipients, or stabilizers. The pharmaceutical compositions can be in solid or liquid form such as, tablets, capsules, powders, solutions, suspensions, or emulsions, and can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by introduction into one or more lymph nodes. For most therapeutic purposes, peptides or nucleic acids can be administered intravenously or parenterally.

For injectable dosages, solutions or suspensions of the one or more therapeutic agents can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are liquid carriers, particularly for injectable solutions.

For use as aerosols, the one or more therapeutic agents in solution or suspension may be packaged in a pressurized aerosol container together with suitable propellants, for example, hydrocarbon propellants like propane, butane, or isobutane with conventional adjuvants. The materials also may be administered in a non-pressurized form such as in a nebulizer or atomizer.

### Kits

Another aspect of the present application relates to kits for treating a subject at risk of, susceptible to or suffering from sepsis. The kit comprising C1q plus any other agents packaged in a suitable container and instructions for using the kit.

In one embodiment, the kit comprises an antibody chip and means for cell isolation. In specific embodiments, the kit may comprise a test strip that can show a positive signal when C1q level is above a threshold level (and a negative result would indicate likelihood of onset of sepsis and need for treatment). In specific embodiments, the kit comprises reagents for lysing cells, as well as materials and apparatus to perform ELISA to detect C1q. In some embodiments, the kit further comprises reagents for measuring C1q levels by flow cytometry.

In another embodiment, the kit further contains a dispenser, such as a syringe or an inhaler, for administering C1q plus any other agents.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

C1q protein level is measured in isolated neutrophils. Neutrophils may be isolated according to any method known to one of ordinary skill in the art. For example, blood was collected from healthy volunteers via antecubital vein puncture in heparin-containing vacutainers. The granulocytes and erythrocytes were separated from the whole blood by centrifugation through a one-step polymorphs (Fresenius Kabi Norge AS) density gradient. The remaining erythrocytes were removed by hypotonic lysis, yielding a neutrophil purity of 98%. C1q protein level generally peaks at 3-7 days after sepsis diagnosis.

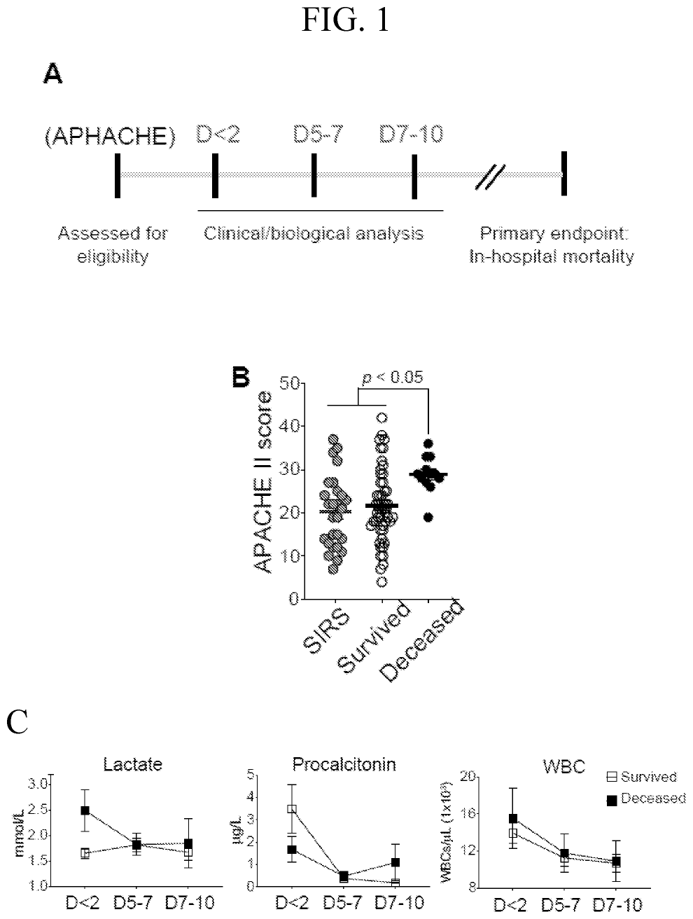

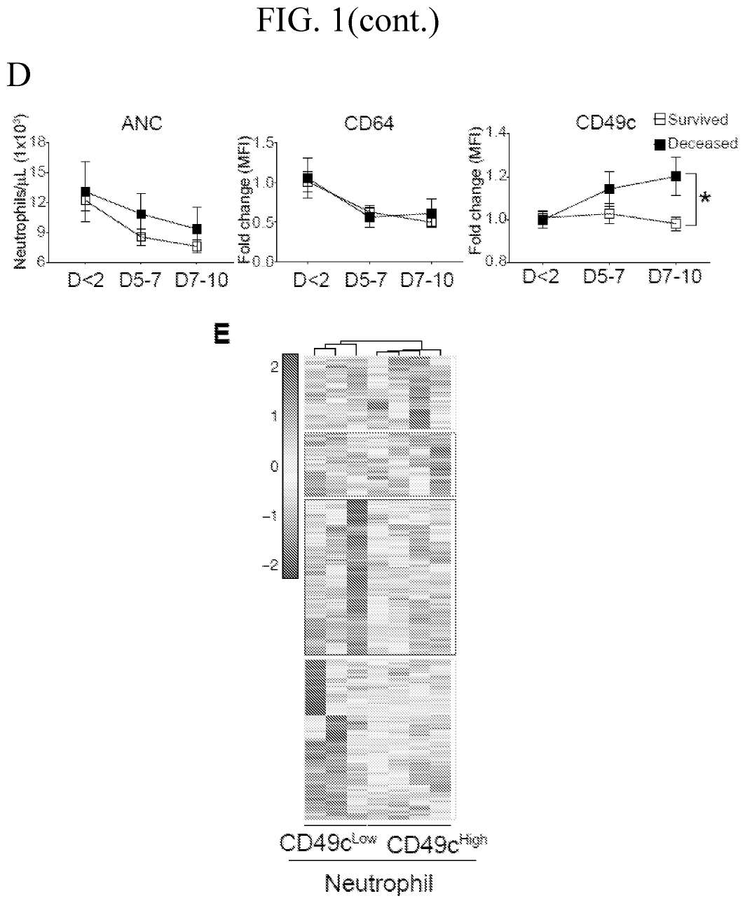

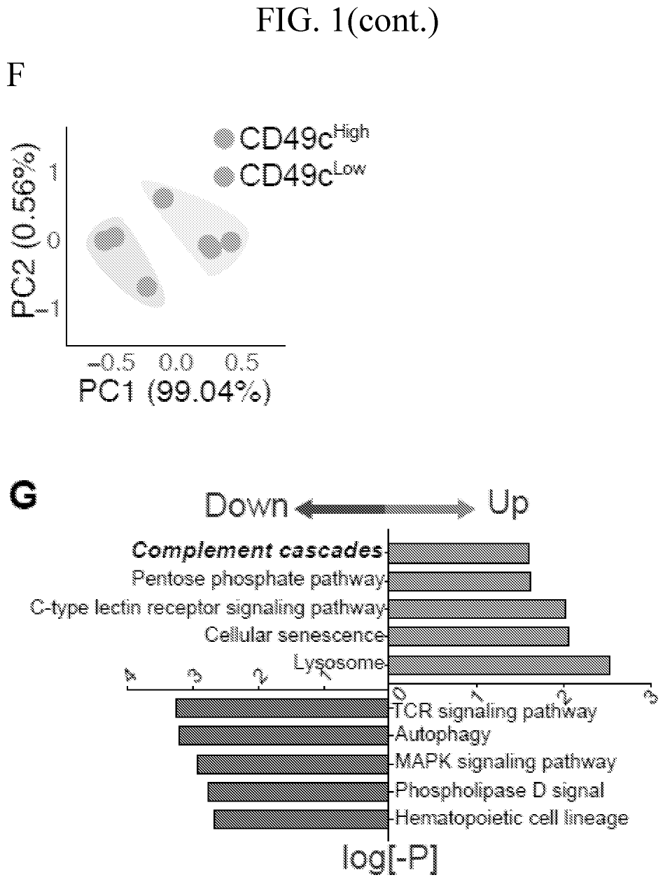

To determine if the routine clinical measurements reflects and predicts severity of illness in ICU septic patients over time, a longitudinal study was performed over the course of their disease. A series of blood donations was obtained from septic patients beginning at the time of diagnosis (within 48 hours of meeting diagnostic criteria) and two subsequent samples at 3-5 day intervals thereafter, along with clinical data including scores of injury severity (ISS, APACHE and SOFA) (FIG. 1, Panel A). Although patients with a final diagnosis of sepsis were based on microbiological data and direct inspection, a significant correlation between the patient's APACHE score and in-hospital mortality independently confirmed the reliability of diagnostic criteria (FIG. 1, Panel B).

Traditional markers for severe infection and sepsis include temperature, leukocytosis, lactate, and procalcitonin (PCT). Although serum levels of lactate, PCT, and WBC were elevated during the early stage of sepsis, none of these inflammation markers reliably predicted survival or death of septic patients (FIG. 1, Panel C). Recently, attention has been directed to the leukocyte cell surface antigens as diagnostic markers of sepsis. Among them, neutrophil surface CD64, the high-affinity Fc receptor, is quantitatively upregulated during infection and sepsis. However, both absolute neutrophil count (ANC) and CD64 expression were not able to predict hospital mortality in septic patients (FIG. 1, Panel D). The data from sepsis patients suggest that those traditional biomarkers may help diagnosis of disease and decrease unnecessary treatments, but do not improve diagnostic ability in determining which patients have more severe immune dysregulation, and thus higher mortality.

The presence of a hyperactive subset of neutrophils that express high levels of CD49c (VLA-3; α3β1) in sepsis patients was observed; and elevated levels of neutrophil CD49c correlated with sepsis diagnosis. Unexpectedly, examination of neutrophils isolated from septic patients revealed that the subpopulation of neutrophils that arises in septic patients was closely associated with sepsis mortality (FIG. 1, Panel D). The results indicate the unique predictive value of the CD49c^{high} neutrophil subpopulation even when sampled later in the course of disease.

Identifying a subpopulation of neutrophils that can predict sepsis outcomes prompted further investigations to discover more reliable functional classifiers for sepsis-associated fatality. First, the transcriptional signature of CD49c^{high} vs CD49c^{low} neutrophils isolated from septic patients by RNAseq was defined. Principal component analysis of the identified genes differentially expressed in septic neutrophils revealed clear transcriptional differences in CD49chigh vs CD49clow neutrophils from patients (FIG. 1, Panel F). Among the 53,736 genes identified, 1,077 were differentially expressed in CD49chigh neutrophils (FIG. 1, Panel E). Pathway analysis revealed that sepsis led to a significant upregulation of genes related to innate inflammatory response in CD49chigh neutrophils, as expected (FIG. 1, Panel G). Interestingly, of those, robust upregulation of several key complement pathway genes was further validated.

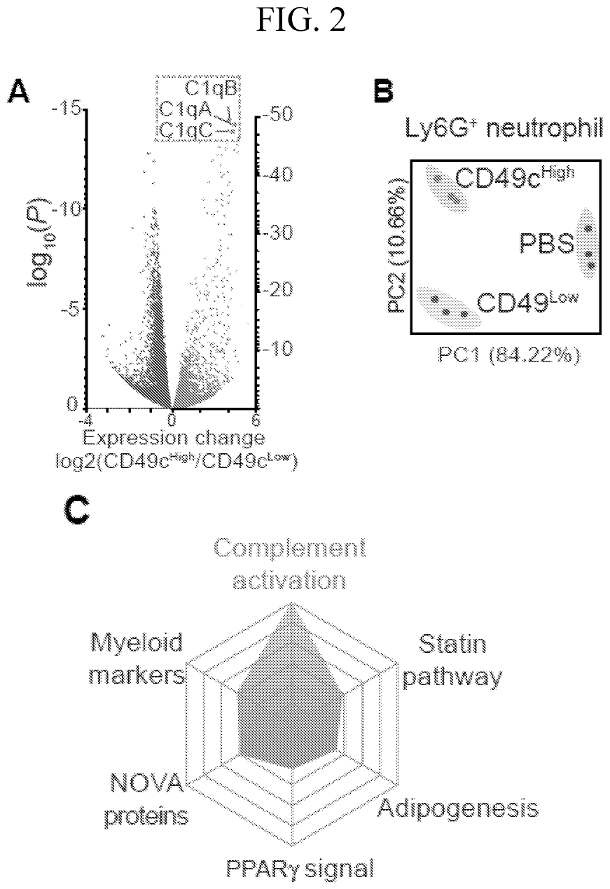

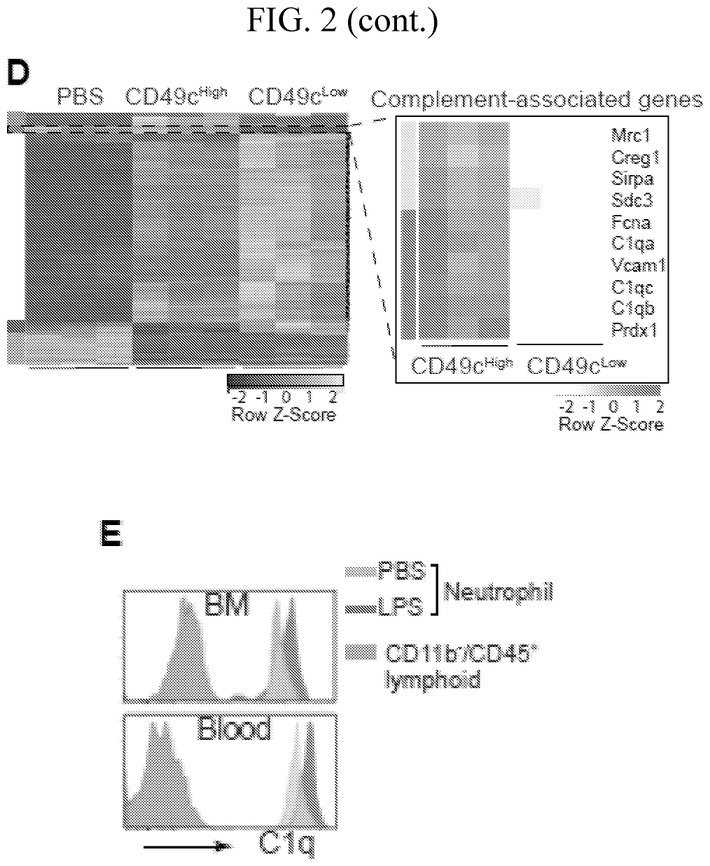

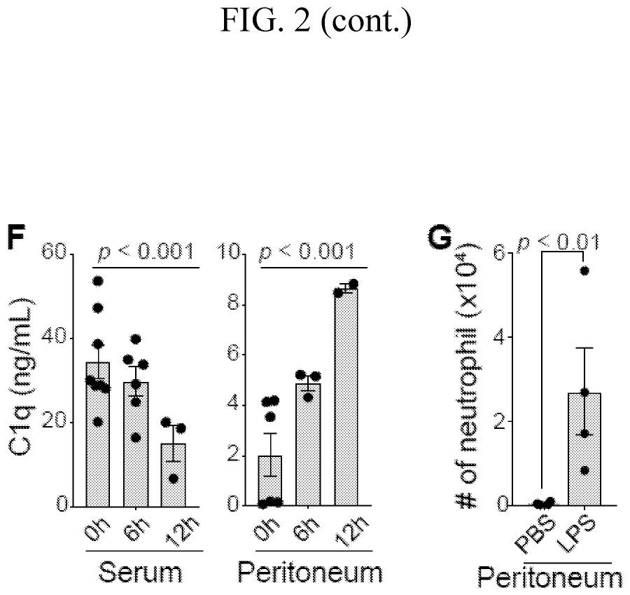

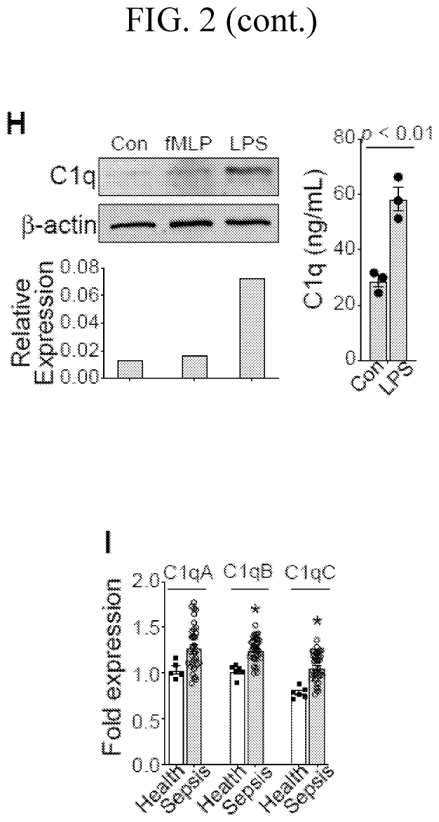

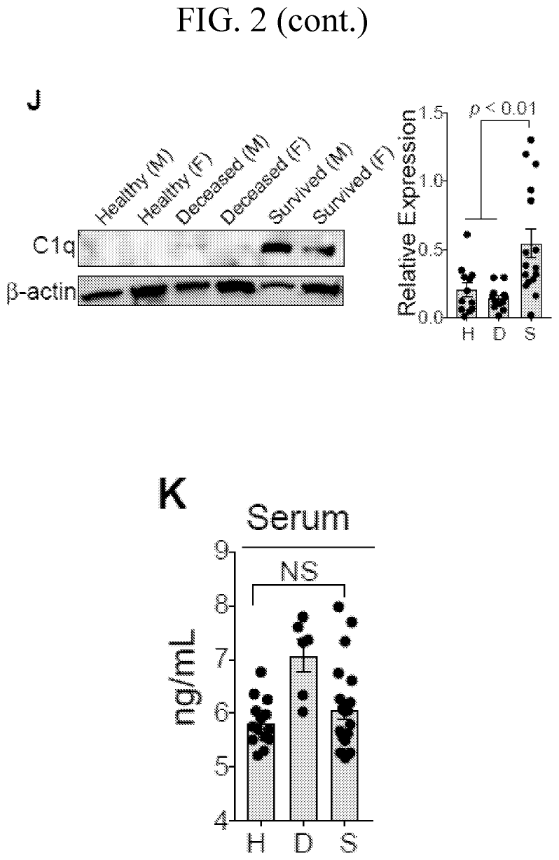

To further verify the gene expression results in a controlled mouse sepsis model, neutrophils from endotoxemic mice were FACS-sorted into CD49c^{high} and CD49c^{low} neutrophil populations, and mRNA was analyzed by RNAseq. Principal component analysis (FIG. 2, Panel B) showed clear transcriptional separation of the neutrophil subsets in mice, and a volcano plot showed differences among specific genes (FIG. 2, Panel A). Among the 16,384 genes identified, 5,717 were found to be differentially expressed between CD49c^{high} and CD49c^{low} septic neutrophils (FIG. 2, Panel D). Of these genes, we identified 326 genes that were significantly overexpressed in CD49c^{high} neutrophils compared to CD49c^{low} neutrophils. Using Enrichr analysis, we ranked these genes according to their protein function category. Remarkably, we found that CD49c^{high} expression was highly correlated with genes in the complement and coagulation cascade (FIG. 2, Panel C), thus confirming our observations in sepsis patient samples (FIG. 1). Among the complement and coagulation cascade genes, the most differentially expressed gene was C1q genes, encoding C1qA, C1qB, and CqC chains that assemble into the complement factor C1q (FIG. 2, Panel A & Panel D).

### Example 2: Flow Cytometry Analysis Further Confirmed a Significant Expression of C1q in Neutrophils from Both naïve and LPS-treated Mice

Flow cytometry analysis further confirmed a significant expression of C1q in neutrophils from both naïve and LPS-treated mice (FIG. 2E). In vivo, serum levels of C1q significantly declined during the first 6-12 hr after LPS-induced mouse endotoxemia, while there was an increase in C1q expression at the peritoneal site of inflammation (FIG. 2, Panel F). Notably, the elevated local C1q level coincided with a massive infiltration of neutrophils in the peritoneum (FIG. 2, Panel G).

In isolated human neutrophils, stimulation with LPS, but not fMLP, significantly elevated expression levels of C1q (FIG. 2, Panel H). Quantitative PCR with reverse transcription (RT-qPCR) analyses revealed that the genes associated with C1q expression significantly increased in neutrophils isolated from the peripheral blood of septic patients, irrespective of the survival status of the individual (FIG. 2, Panel I). Strikingly, only the neutrophils from sepsis patients who survived were able to translate and maintain intracellular C1q protein, as compared with the patients who died (FIG. 2, Panel J). All sepsis patients retained the ability to express C1q transcripts, but poorer patient outcomes were associated with the loss of C1q translation. Importantly, any significant differences in the serum C1q levels in these healthy and patient groups were not observed (FIG. 2, Panel K). Therefore, the data suggest that better sepsis prognosis is associated with the neutrophil specific C1q expression, and that patients who are unable to produce C1q protein in neutrophils have a higher likelihood of sepsis mortality.

### Example 3: C1q Have a Role Outside of the Classical Complement Cascade, Functioning as an Important Inflammatory Mediator During Severe Systemic Inflammation

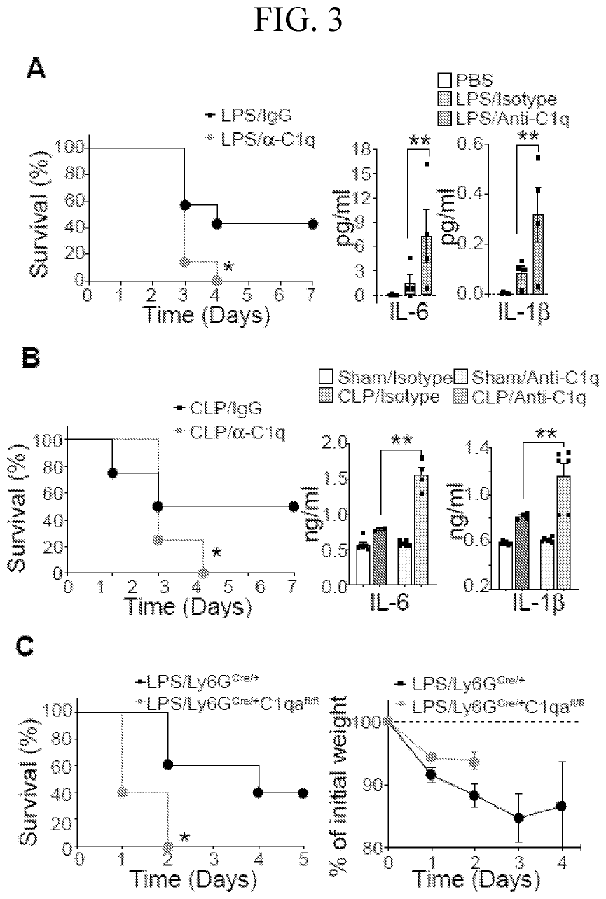

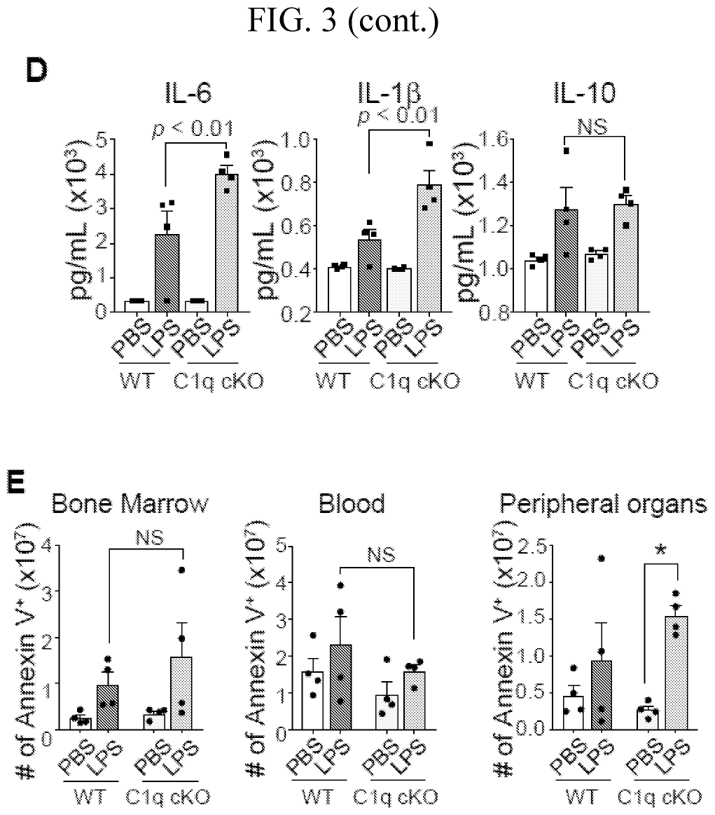

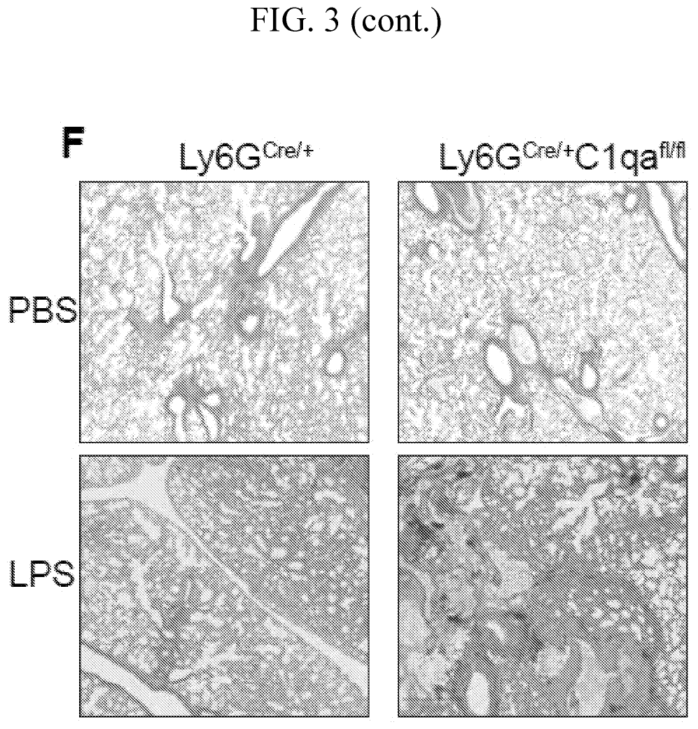

C1q is the initiator molecule in the classical complement cascade, which ultimately leads to cell lysis by the formation of the membrane attack complex (MAC). In addition to the conventional complement pathways, C1q function as a soluble pattern recognition molecule that binds to IgG- or IgM-containing immune complexes, where it then recruits serine proteases C1r and C1s to immobilize on the surface of microorganisms or danger associated molecular patterns expressed on a host cell. The presence of a high C1q level in the inflamed tissue and increased neutrophil C1q production in sepsis survivals led us to hypothesize that C1q have a role outside of the classical complement cascade, functioning as an important inflammatory mediator during severe systemic inflammation and that secretion of C1q locally at the inflamed tissue sites by newly recruited neutrophils may be critical for the patient survival during sepsis. To test this hypothesis, a C1q neutralizing antibody was administered to the mouse peritoneum 1 hr prior to septic challenges. For this functional assay, the following two mouse models were used: LPS-induced endotoxemia, which can bypass the need for C1q to bind to antibody coated bacteria and initiate the complement cascade, and Cecum Ligation and Puncture (CLP) surgery. Blocking of C1q function locally at the site of inflammation dramatically increased sepsis mortality and significantly increased pro-inflammatory cytokine levels IL-6 and IL-1b in the serum in both mouse sepsis models (FIG. 3, Panel A & Panel B). Importantly, the local injection of C1q neutralizing antibody did not alter the systemic level of C1q in the serum.

Determining the contributions of C1q to immune functions in vivo has been challenging because of the increased mortality of a C1q subunit knockout in mice, which die of severe autoimmunity by an impaired clearance of apoptotic cells. To circumvent this problem and to further assess the function of neutrophil-derived C1q on sepsis, a conditional knockout C1qflox/flox; Ly6G-Cre (C1q cKO) was generated by crossing C1qa floxed mice with a mouse line in which the first exon of the Ly6g gene is replaced by a knock-in allele encoding Cre recombinase. Deletion of the floxed a subunit alleles and the absence of protein expression were confirmed by PCR and western blot analysis. C1qwt/wt; Ly6G-Cre (WT) mice as littermate controls was used. Although C1q cKO mice showed similar severity of inflammation as their WT littermates during the early period a mild endotoxemia, the absence of neutrophil-derived C1q resulted in a dramatically increased mortality compared with the control WT mice after LPS treatments (FIG. 3, Panel C). Serum levels of IL-6 and IL-1b were significantly elevated in the C1q cKO mice compared with the controls, indicating the increased severity of sepsis (FIG. 3, Panel D). On the other hand, neutrophil development, bacterial clearance, TLR4 surface expression level, and inflammation mediated oxidative burst by neutrophils isolated from C1q cKO mice were unaffected. The increased mortality in C1q neutralizing antibody-treated mice and C1q cKO mice was likely due to the accumulation and/or delayed clearance of apoptotic neutrophils in the inflamed lungs and the peritoneal space, as measured within 24 hr of endotoxemia (FIG. 3, Panel E). FIG. 3, Panel F shows lung tissue sections in C1q cKO mice and the controls.

### Example 4: Local C1q Secretion by Apoptotic Neutrophils is Necessary for Their Prompt Efferocytosis and Clearance by Phagocytes

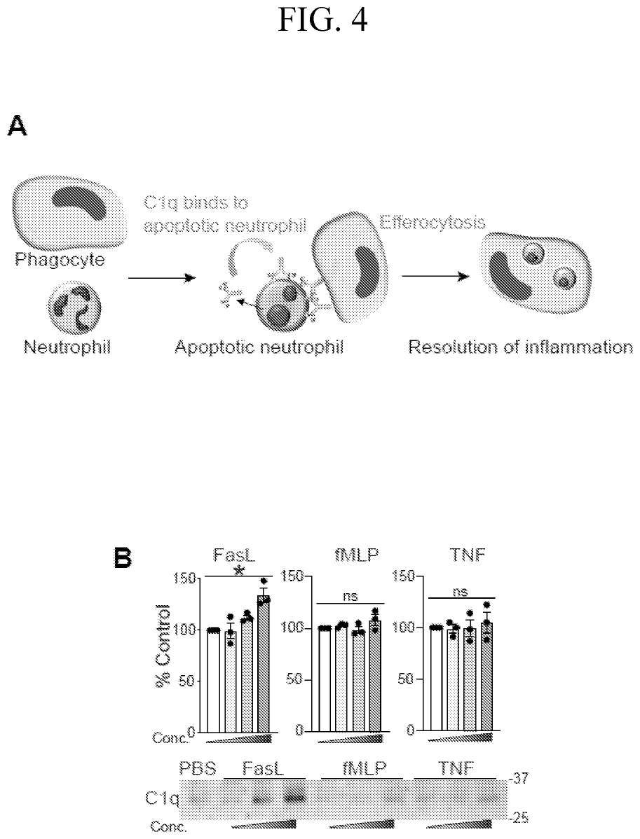

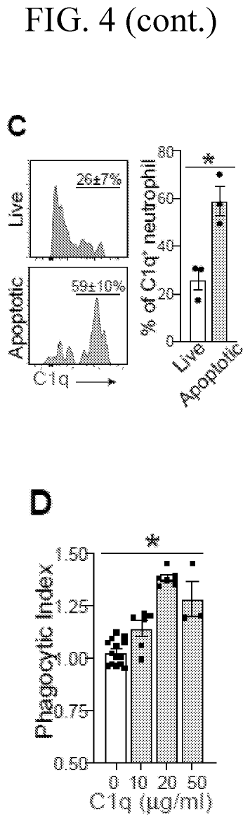

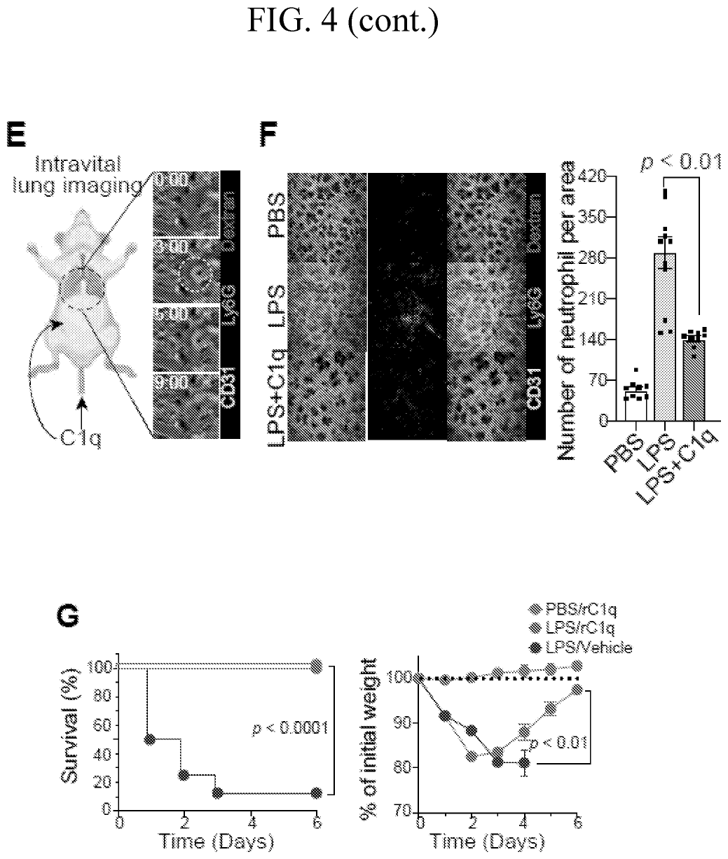

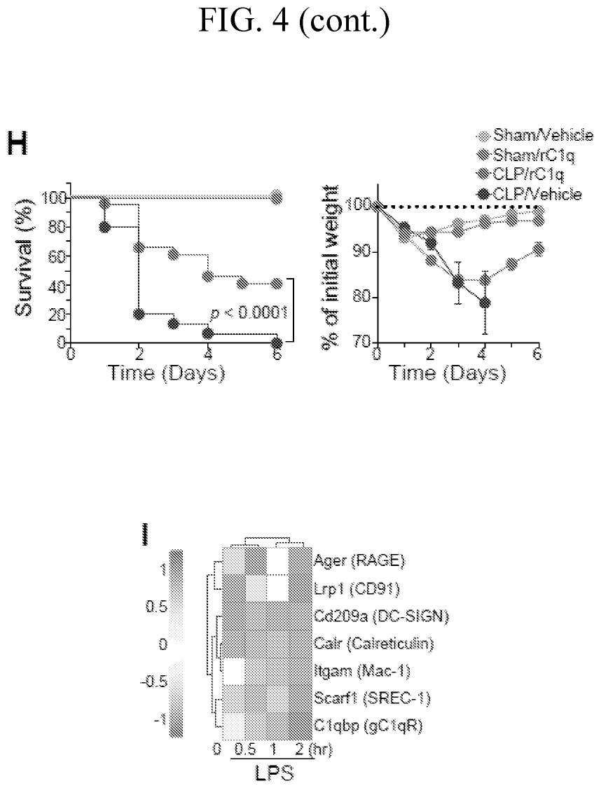

Recruitment of neutrophils from blood to sites of tissue infection is vital for early innate immune responses during sepsis. Once they complete their action, infiltrated neutrophils should quickly initiate spontaneous apoptosis and cleared from the tissue. A delayed resolution of neutrophil response is often associated with widespread tissue damage, organ failure, and ultimately death in critically ill patients. Therefore, the presence of unresolved neutrophil response has long been believed to be detrimental. C1q binds to phosphatidylserine (PS) on apoptotic cells and mediates efferocytosis. Therefore, it was hypothesized that local C1q secretion by apoptotic neutrophils is necessary for their prompt efferocytosis and clearance by phagocytes, which is essential for better patient prognosis during sepsis (FIG. 4, Panel A). To investigate whether neutrophil secrets C1q in response to inflammatory stimulations, western blot analysis of neutrophil supernatants was performed. Interestingly, even with high concentrations of inflammatory stimuli that induce neutrophil degranulation such as TNF or fMLP, there was a failure to detect a significant release of soluble C1q from neutrophils (FIG. 4, Panel B). To screen for potential stimulators that trigger C1q release from neutrophils, next, neutrophil apoptosis was studied. The secretion of C1q was most significantly enhanced after the induction of neutrophil apoptosis with Fas ligand (FasL) (FIG. 4, Panel B).

If the secretion of C1q from neutrophils depends on cell apoptosis, conceptually it may be possible that the neutrophil-derived C1q functions as an important ‘eat me’ signal to promote efferocytosis by local phagocytes during the resolution of septic inflammation and that this signal is a key for the patient survival. Flow cytometry analyses of live vs. apoptotic neutrophils further revealed a greater extend of C1q binding on the apoptotic neutrophil surface (FIG. 4, Panel C). In addition, C1q showed a significant dose-dependent enhancement of the phagocytic uptake of apoptotic neutrophils by macrophages (FIG. 4, Panel D). Importantly, loss of intrinsic C1q expression did not alter the apoptotic frequency of neutrophils.

In a mouse model of sepsis-induced acute lung injury (ALI), massive tissue infiltration and subsequent sequestration of unresolved neutrophils in the pulmonary microcirculation leads to accompanying acute respiratory distress syndrome (ARDS). Indeed, intravital multiphoton microscopy (IV-MPM) of the mouse lung with a custom-made pulmonary imaging window revealed a significant number of neutrophil aggregates in the pulmonary microcirculation of the LPS-induced ALI mice (FIG. 4, Panel E). Note that the unresolved neutrophil aggregates were a main cause of the pulmonary capillary obstruction in sepsis-induced ALI mice. In these LPS-induced ALI mice, administration of C1q (20 mg×3, IP) significantly reduced the number of neutrophil aggregates in mice 24 hr after LPS stimulation (FIG. 4, Panel F). These findings parallel the mouse survival assays, where periodic C1q injection protected mice from sepsis lethality both in the LPS-induced endotoxemia model and the CLP surgery model of sepsis (FIG. 4, Panel G & Panel H)

The finding that apoptotic neutrophils secrete C1q and “self-decorate” in an autocrine and/or paracrine fashion, marking them for efficient efferocytosis suggests that neutrophil is a main source of tissue C1q (not serum C1q) that mediate apoptotic neutrophil efferocytosis by tissue-resident macrophage leading to successful resolution of inflammation and improved septic survival. Indeed, among the potential C1q receptors known so far, peritoneal macrophages isolated from LPS-treated mice upregulated at least five of the receptors (FIG. 4, Panel F), suggesting that the apoptotic neutrophil-macrophage interaction during sepsis is at least partly mediated by these receptors

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it, which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A method of treating sepsis or a sepsis-related condition, comprising the step of:
administering to a subject in need of such treatment an effective amount of C1q protein or a variant thereof.

2. The method of claim 1, wherein the subject is diagnosed with sepsis and wherein C1q protein expression in neutrophils of the subject is below a threshold level.

3. The method of claim 1, further comprising the step of: administering to the subject an antibiotic.

4. The method of claim 1, wherein the C1q protein or a variant thereof is administered by inhalation, intra-tracheal delivery, nebulized and inhaled delivery, intraurethral delivery, intravenous injection or combinations thereof.

5. The method of claim 1, wherein the C1q protein or a variant thereof is administered in an amount of 0.1-10 mg/kg body weight daily for a period of 1-14 days.

6. The method of claim 5, wherein the C1q protein or a variant thereof is administered in an amount of 0.3-3 mg/kg body weight daily for a period of 2-7 days.

7. The method of claim 5, wherein the C1q protein or a variant thereof is administered in an amount of 0.1-10 mg/kg body weight three time a day by local injection or intravenous injection or a combination of both for a period of 2-7 days.

8. The method of claim 1, wherein the C1q protein or a variant thereof is administered by inhalation for treatment of sepsis caused by lung infection.

9. The method of claim 1, wherein the C1q protein or a variant thereof is administered by retrograde infusion from bladder for treatment of sepsis caused by kidney infection.

10. The method of claim 1, wherein the C1q protein or a variant thereof is administered by intraurethral delivery for treatment of sepsis caused by bladder infection.

11. The method of claim 1, wherein the sepsis-related condition is selected from the group consisting of pneumonia, pneumonitis, urinary tract infection, peritonitis, infections of the biliary system including cholecystitis, cholangitis, colitis, enteritis, bowel obstruction, bowel perforation, bloodstream infection, meningitis, encephalitis, cellulitis, skin and soft tissue infections, prostatitis, endometritis, and post-operative wound infections.

12. A method of determining sepsis with poor prognosis in a subject, said method comprising:
isolating neutrophils from the subject;
determining a level of C1q protein in the isolated neutrophils, and
administering C1q protein or a variant thereof to the subject, if the level of C1q protein is below a pre-determined threshold.

13. The method of claim 12, wherein the neutrophils are isolated from the blood of the subject.

14. The method of claim 12, wherein the level of C1q is determined by ELISA.

15. The method of claim 12, wherein the subject is a mammal.

16. A kit for providing a prognosis of a subject with sepsis, comprising:
a test device for determining a level of C1q in a test sample; and
a user guide.

17. The kit of claim 16, wherein the test device is a testing strip that provides a visible signal when in contact with the test sample and when the level of C1q in the test sample is above a threshold level, and wherein a negative test result indicates a poor prognosis.

18. The kit of claim 16, further comprising reagents for measuring C1q levels by flow cytometry.

19. The kit of claim 16, further comprising a cell lysis reagent.

20. A pharmaceutical composition, comprising:
a recombinant C1q protein or a variant thereof; and
a pharmaceutically acceptable carrier,
wherein the pharmaceutical composition is formulated for inhalation.
